(19) United States
(12) Patent Application Publication
(10) Pub. No.: US 2026/0102212 A1
(43) Pub. Date: Apr. 16, 2026

(54) System And Method For Implantable Sensor Registration

(71) Applicant: Howmedica Osteonics Corp., Mahwah, NJ (US)

(72) Inventors: Matthias Verstraete, Chaam (NL); Andrew Meyer, Plantation, FL (US)

(73) Assignee: Howmedica Osteonics Corp., Mahwah, NJ (US)

(21) Appl. No.: 19/120,637

(22) PCT Filed: Oct. 13, 2023
(86) PCT No.: PCT/US2023/035075

§ 371 Date: Apr. 11, 2025

Related U.S. Application Data
(60) Provisional application No. 63/379,384, filed on Oct. 13, 2022.

(51) Int. Cl. A61B 34/20; A61B 5/107; A61B 5/11

(52) U.S. Cl. CPC A61B 34/20; A61B 5/1071; A61B 5/1114; A61B 2034/2059; A61B 2034/2065

(57) ABSTRACT

A method for registering a sensor coordinate system of a first sensor of an implant to an anatomical coordinate system using a surgical system is provided. The implant includes a second sensor, and the surgical system includes a surgical navigation system. The first sensor has a predetermined spatial relationship relative to the implant when the implant is coupled to a bone of a patient. The method includes receiving at least one medical image defining the anatomical coordinate system, the at least one medical image including a first body segment and a second body segment forming a joint. In addition, the method includes determining a first angle of the joint based on motion data, determining a second angle of the joint based on surgical navigation system, comparing the first angle to the second angle, and selectively adjusting one or more parameters of the first sensor.

### CROSS-REFERENCE TO RELATED APPLICATIONS

This application claims the benefit of and priority to U.S. Provisional Patent Application No. 63/379,384, filed Oct. 13, 2022, which is incorporated herein by reference in its entirety.

### BACKGROUND

Replacement arthroplasty or joint replacement surgery is commonly performed to replace a damaged joint with an orthopedic implant. One such procedure commonly performed by medical professionals is a total knee arthroplasty (TKA) or partial knee arthroplasty (PKA) in which a medical professional replaces all or part of a damaged knee joint of a patient with an implant. In a TKA procedure, the medical professional may replace the weight bearing surface of the tibia with a tibia component and the weight bearing surface of a femur with a femoral implant. In a PKA procedure, the medical professional may only replace one of the weight bearing surface of the tibia and the weight bearing surface of the femur.

The implant may include one or more sensors, such as an inertial measurement unit (IMU) sensor and/or hall-effect sensor, which may provide motion data which is used to track various parameters associated with a patient such as range of motion by measuring a joint angle, step count, fall detection, alignment of the implant components, wear and tear and the like. After the joint arthroplasty surgery is performed, a patient may complete a physiotherapy program to for several months post-surgery. The motion data may be used to track the recovery of a patient throughout physiotherapy program and even after the physiotherapy program.

In order to output motion data, IMUs include sensors such as gyroscopes, accelerometers, and/or magnetometers. These sensors measure and output kinematic/motion data relative to axes associated with the sensor, these axes forming a sensor coordinate system. On the other hand, the aforementioned parameters (e.g. joint angle) are most useful when tracked in a coordinate system associated with the patient, such as an anatomical coordinate system. Therefore, it may be necessary to determine an association between the sensor coordinate system and the anatomical axis. In the art, determining said association is referred to as “registration”. Such registration is most easily achieved when the pose of the IMU coordinate system is known relative to the pose of the anatomical coordinate system. Ordinarily, IMUs are visible and/or have a known pose, however, IMUs included in an implant are often not visible nor in a known orientation once the implant is implanted in the patient. Thus, a registration between the patient's anatomical coordinate system and the IMU coordinate system is difficult to perform. As such, a system capable of overcoming the above-mentioned challenges is desirable.

### SUMMARY

In one aspect, a method for registering a sensor coordinate system of a first sensor of an implant to an anatomical coordinate system using a surgical system is provided. The implant includes a second sensor, and the surgical system includes a surgical navigation system. The first sensor has a predetermined spatial relationship relative to the implant when the implant is coupled to a bone of a patient. The method includes receiving at least one medical image defining the anatomical coordinate system, the at least one medical image including a first body segment and a second body segment forming a joint. After the implant has been coupled to the first body segment and the second body segment forming the joint, while the first body segment and the second body segment are in a first pose, the method includes obtaining motion data from at least one of the first sensor and the second sensor and obtaining navigation data of the first body segment and the second body segment with surgical navigation system. In addition, the method includes determining a first angle of the joint based on the motion data, determining a second angle of the joint based on the surgical navigation system, comparing the first angle to the second angle, and selectively adjusting one or more parameters of the first sensor so that the first angle falls within a threshold of the second angle.

In another aspect, a system for registering a sensor coordinate system of a sensor of an implant to an anatomical coordinate system using a surgical system is provided. The system includes the implant including a sensor defining a sensor coordinate system including at least one sensor axis. The implant is configured to be fixed to a first bone of a patient and the sensor has a first fixed spatial relationship relative to the implant. The sensor is configured to output inertial data of the implant. The system further includes a patient tracking element coupled to the patient, and a surgical instrument configured to couple to the implant. The surgical instrument includes an instrument tracking element. The system further includes a navigation system, and the navigation system is configured to receive an image of the patient defining an anatomical coordinate system, register the patient in a global coordinate system to the anatomical coordinate system, track a pose of the surgical instrument in the global coordinate system, determine a pose of the implant based on the pose of the surgical instrument in the global coordinate system, determine the pose of the implant in the anatomical coordinate system, and selectively adjust one or more parameters of the sensor to register the sensor coordinate system to the anatomical coordinate system.

In yet another aspect, a system for registering a sensor coordinate system of a sensor of an implant to an anatomical coordinate system using a surgical system is provided. The system includes the implant including a sensor defining a sensor coordinate system including at least one sensor axis, and the implant is configured to be fixed to a bone of a patient. The sensor has a first predetermined fixed spatial relationship relative to the implant. An implant attachment element is provided which is configured to be attached to the implant, the implant attachment element including a tracking element and having a second predefined spatial relationship with the implant when the implant attachment element is coupled to the implant. The system further includes a patient tracking element configured to be attached to the patient and a navigation system. The navigation system is configured to receive an image of the patient defining an anatomical coordinate system, register the patient in a global coordinate system to the anatomical coordinate system, track a pose of the implant attachment element in the global coordinate system, determine a pose of the implant based on the pose of the implant attachment element in the global coordinate system, determine the pose of the implant in the anatomical coordinate system, and selectively adjust one or more parameters of the sensor to register the sensor coordinate system to the anatomical coordinate system.

In yet another aspect, a system for registering a sensor coordinate system of a sensor of an implant to an anatomical coordinate system using a surgical system is provided. The system includes an implant including a sensor defining a sensor coordinate system. The sensor includes at least one sensor axis, and the implant is configured to be fixed to a bone of a patient. Further, the sensor has a predetermined fixed spatial relationship relative to the implant. The system further includes a surgical robot configured to remove a target volume of a bone, and a navigation system. The navigation system is configured to receive a surgical plan including a planned pose of an implant relative to a pre-op image, receive at least one parameter of the implant, the at least one parameter of the implant being indicative of a predetermined fixed spatial relationship relative to the implant, remove a target volume of the bone based on the planned pose of the implant. The navigation system is further configured to guide the surgical robot to install the implant based on planned pose of the implant, determine a pose of the sensor relative to an anatomical coordinate system based on the planned pose for the implant, and selectively adjust one or more parameters of the pose of the sensor relative to the anatomical coordinate system to register the sensor coordinate system to the anatomical coordinate system.

In yet another aspect, a system for registering a sensor coordinate system of a sensor of an implant to an anatomical coordinate system using a surgical system is provided. The system includes an implant including a sensor defining a sensor coordinate system including at least one sensor axis. The implant is configured to be fixed to a bone of a patient, and the sensor has a predetermined fixed spatial relationship relative to the implant. The system further includes a surgical instrument configured to remove a target volume of the bone, and a navigation system. The navigation system is configured to receive a surgical plan including a planned pose of an implant relative to a pre-op image, receive at least one parameter of the implant, the at least one parameter of the implant being indicative of predetermined fixed spatial relationship relative to the implant. The navigation system is further configured to determine a pose of the sensor relative to an anatomical coordinate system based on the planned pose of the implant, and selectively adjust one or more parameters of the pose of the sensor relative to the anatomical coordinate system to register the sensor coordinate system to the anatomical coordinate system.

In yet another aspect, a method for registering a first coordinate system of a first sensor to an anatomical coordinate system is provided. The first sensor is coupled to an implant, and the implant includes a first component and a second component. Additionally, the first sensor is configured to output first motion data, and the first sensor is coupled to the first component while a second sensor is coupled to the second component. The second sensor defines a second coordinate system and is configured to output second motion data. The first component is coupled to a first bone of a patient and the second component is coupled to a second bone, the first bone and the second bone forming a joint. The method includes receiving one or more biomechanical constraints of the joint of the patient, collecting a first motion data the first sensor and a second motion data from the second sensor while the patient moves from a first pose to a second pose, determining at least one transform between the first coordinate system of the first sensor and the anatomical coordinate system based on at least one of the first motion data, the second motion data, the biomechanical constraints of the joint, and the second coordinate system. Finally, the method includes selectively adjust one or more parameters of the first sensor based on the determined at least one transform to register the first coordinate system to the anatomical coordinate system.

In yet another aspect, a system for registering a sensor coordinate system of a sensor of an implant to an anatomical coordinate system using a surgical system is provided. The system includes an implant including a first component configured to couple to a first bone of a patient and a second component being configured to couple to a second bone of the patient, a joint of the patient being formed where the first bone and the second bone meet. The system further includes a first sensor configured to output a first motion data and defining a first coordinate system including at least one first sensor axis. Wherein the first sensor is coupled to the first bone, and the first sensor has a first predetermined fixed spatial relationship relative to the first component. The system further includes a second sensor configured to couple to the second component. The second sensor defines a second coordinate system and is configured to output a second motion data. The system further includes a navigation system. The navigation system is configured to receive one or more biomechanical constraints of the joint of the patient, collect the first motion data from the first sensor and the second motion data from the second sensor while the patient moves from a first pose to a second pose, and determine at least one transform between the first coordinate system of the first sensor and an anatomical coordinate system based on at least one of the first motion data, the second motion data, the biomechanical constraints of the joint, and the second coordinate system. Finally, the system is configured to selectively adjust one or more parameters associated with the first sensor based on the determined at least one transform to register the first coordinate system to the anatomical coordinate system.

In yet another aspect, a method for registering a sensor coordinate system of a sensor coupled to an implant is provided. The sensor coordinate system includes at least one sensor axis, and the implant includes a first component and a second component. The first component is configured to be coupled to a first bone of a patient and the second component is configured to be coupled to a second bone of the patient. The sensor has a fixed spatial relationship relative to the first component. The method includes receiving a model, wherein the model includes the first bone and the second bone and the model is generated based on at least one pre-operative image of the first and second bone. Additionally, the model defines a pose of a planned implant relative to the first bone and the second bone, and the model defines at least one anatomical coordinate system. The method further includes receiving at least one post-operative image including an artifact of the implant, aligning the post-operative image to the model, determining a pose of the implant in the anatomical coordinate system based on the aligned post-operative image to the model, determining at least one transform between the sensor coordinate system of the sensor and the anatomical coordinate system based on the determined pose of the implant and at least one anatomical axis defined in the anatomical coordinate system, and selectively adjusting one or more parameters associated with the first sensor based on the determined at least one transform to register the first sensor coordinate system to the anatomical coordinate system.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 depicts an implant including a femoral component and a tibia component including a sensor, the femoral component being coupled to a femur and the tibial component being coupled to the tibia and one or more axes of the femur and tibia.

FIG. 2 is a perspective view of surgical system including a robotic system.

FIG. 3 is a schematic view of a control system of the surgical system.

FIG. 4 is an illustration of various transforms used in navigation.

FIG. 5 depicts an implant including a femoral component with a plurality of magnets and a tibial component with an implant module.

FIG. 6 depicts a tibia implant tracking attachment configured to couple to a tibial component.

FIG. 7 depicts a femoral implant tacking attachment configured to couple to a femoral component.

FIG. 8 depicts a femoral IMU attachment configured to couple to a femoral component.

FIG. 9 depicts an exemplary method for registering a coordinate system of an IMU to an anatomical coordinate system, according to a first implementation.

FIG. 10 depicts an exemplary method for registering a coordinate system of an IMU to an anatomical coordinate system, according to a second implementation.

FIG. 11 depicts an exemplary method for registering a coordinate system of an IMU to an anatomical coordinate system, according to a third implementation.

FIG. 12 depicts an exemplary method for registering a coordinate system of an IMU to an anatomical coordinate system, according to a fourth implementation.

FIG. 13 depicts an exemplary method for registering a coordinate system of an IMU to an anatomical coordinate system, according to a fifth implementation.

FIG. 14 depicts an exemplary method for registering a coordinate system of an IMU to an anatomical coordinate system, according to a sixth implementation.

FIG. 15 depicts an exemplary method for registering a coordinate system of an IMU to an anatomical coordinate system, according to a seventh implementation.

In the drawings, reference numbers may be reused to identify similar and/or identical elements.

### DETAILED DESCRIPTION

One challenge of calculating joint angles with inertial measurement units (IMUs) is determining the pose of an IMU sensor on each segment relative to the axis (e.g., anatomical or mechanical axis) of the segment. IMUs may be employed in a wearable manner, stated differently, the IMUs may be attached or otherwise coupled to the outside of a patient. Many methods exist for determining the pose of wearable IMUs relative to the mechanical axis of the segment. For example, some of the methods require a patient to orient his/her body segment at a known static pose, such as in a full joint extension, or require a patient to perform dynamic movements. This is easier for wearable type systems, as the patient is conscious and able to follow instructions given by a medical professional. Additionally, wearable sensors are adjustable should adjustment need to be made to the pose of the wearable sensors.

When measuring joint kinematics, an important consideration is the zero level of each joint. For consistency, joint angles (e.g. flexion/extension (F/E) angle, internal/external (I/E) rotation angle, and/or varus/valgus (V/V) angle) are measured with respect to a pose referred to as “the anatomical position.” In the anatomical position, a person stands straight or lies down flat so that the spine, legs, arms, neck, and head are approximately parallel. The feet point anteriorly, and the palms point anteriorly. The bulk of medical knowledge of human joint kinematics is measured relative to the anatomical position, so devices for measuring joint motion should report angles according to this convention.

IMUs sensors measure linear accelerations and angular velocities. When attached to segments of the human body, these sensors can be used to calculate segment linear motion and angular orientation. When several body segments are recorded simultaneously, the angles of the joints coupling two segments can be calculated.

In the implantable context, there are additional challenges to consider when registering a sensor coordinate system to the anatomical coordinate system of the patient. First, the patient is not conscious and is thus unable to follow directions to perform any specific movement sequence; therefore, any movement sequence that involves any segment of the patient must be capable of being maneuvered by the surgeon. Second, the implant is typically not adjustable once coupled to the patient. And third, the pose of the implant is not easily determined once the implant is implanted into the patient.

IMUs or other sensors such as accelerometers/gyroscopes may be coupled to a patient either directly or indirectly to the patient via an implant to evaluate joint kinematics. In order to provide an accurate evaluation of joint kinematics, such as joint angle, the offset of a coordinate system of the IMU relative to an anatomical coordinate system must be known. Stated differently, a pose of the IMU relative to an axis (e.g., a mechanical axis or an anatomical axis) or joint line must be determined. Once this pose is established the appropriate adjustments may be made to parameters of the IMU so that an accurate joint angle may be calculated based on the motion data.

Disclosed herein is a system and method to determine the alignment of an IMU placed in any location or orientation on one body segment or two adjacent body segments (such as the limb(s) of a mammal). More specifically, the system provided herein allows one to determine the alignment of the coordinate system of the IMU relative to an anatomical coordinate system of said mammal. The teachings of the present disclosure may also be applicable to wearable sensors directly or indirectly coupled to a skin of the patient.

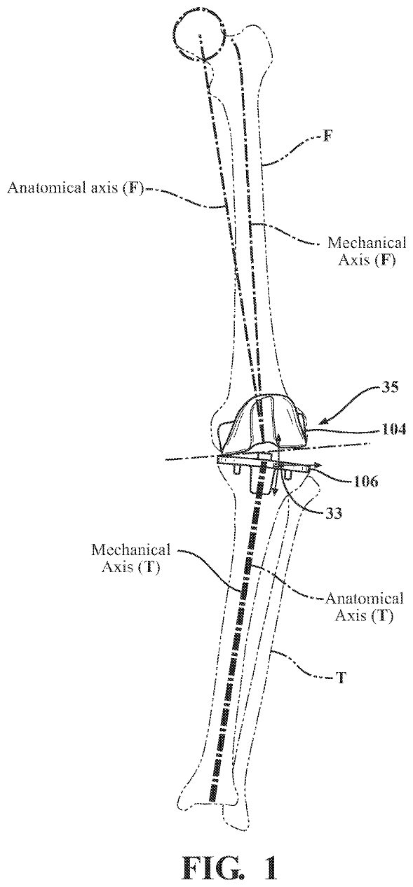

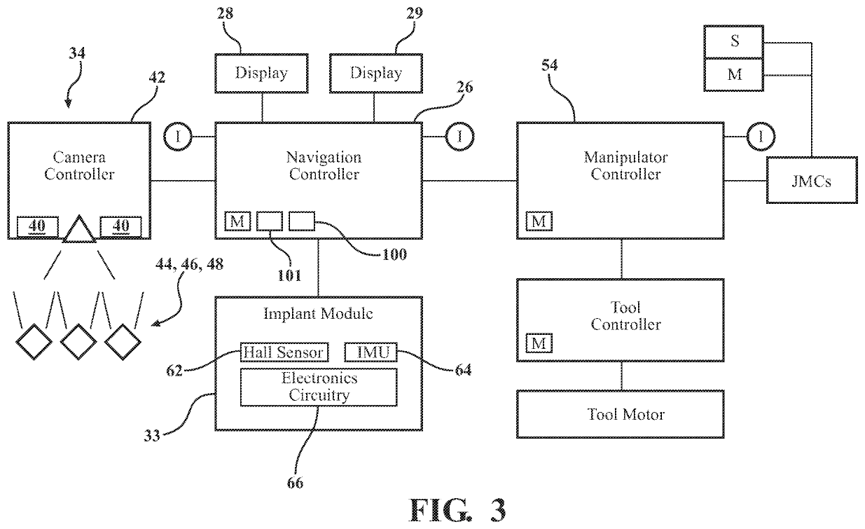

With reference to FIG. 1, the implant 35, as shown, includes a femoral component 104 and a tibial component 106. The femoral component 104 may be coupled to the femur F and the tibial component 106 may be coupled to the tibia T. The implant 35 may further include an identification feature (not shown), and the navigation system 20 may know the pose of the implant 35 based on a pose of the identification feature. The identification feature may be a barcode, a QR code, a physical structure, or anything discernable by the navigation system 20. An implant sensor module 33 may be coupled to or housed within the femoral component 104 and/or the tibial component 106. As will be discussed in greater detail below, with reference to FIGS. 1 and 3, the implant sensing module 33 may include at least one of a hall-effect sensor 62 and an inertial measurement unit (IMU) 64. The sensors 62, 64 may have a fixed pose relative to the geometry of the implant 35. The IMU 64 also defines a sensor coordinate system (e.g., an x-axis, a y-axis, and a z-axis), and raw acceleration/orientation data from the IMU 64 is generally determined by the IMU 64 relative to the sensor coordinate system. For example, raw acceleration/orientation from the IMU 64 may correspond to a roll about the x-axis of the sensor coordinate system, a pitch about the y-axis of the sensor coordinate system, and a yaw about the z-axis of the sensor coordinate system.

The sensor coordinate system of the IMU 64 is typically not aligned with one or more axes of the anatomical coordinate system (e.g., a mechanical axis or an anatomical axis) or lines (e.g., joint line) of the tibia. As a result, one or more parameters (e.g., offset angles/rotations) must be determined which describe the relative pose between one or more axes of the sensor coordinate system of the IMU 64 and one or more axes (e.g., a mechanical axis or an anatomical axis) or lines (e.g., joint line) of the anatomical coordinate system of the tibia T.

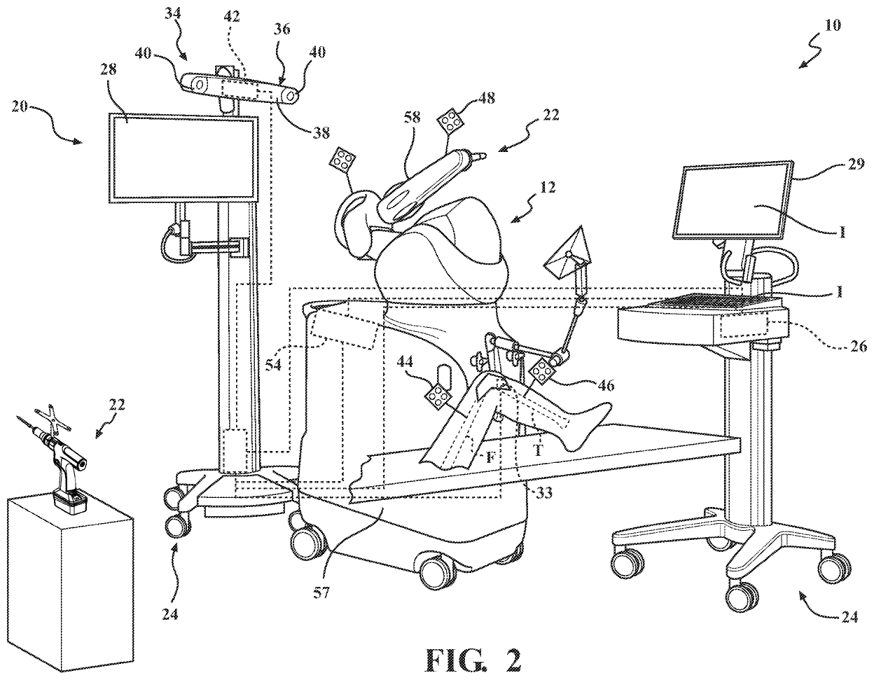

Referring to FIGS. 2 and 3, a surgical system 10 for treating a patient is illustrated. The surgical system 10 is shown in a surgical setting such as an operating room of a medical facility. In the embodiment shown, the system 10 includes a manipulator 12 and a navigation system 20. The navigation system 20 is set up to track movement of various real objects in the operating room. Such real objects include, for example, the manipulator, 12, a surgical instrument 22, a femur F of a patient, a tibia T of the patient, and the implant 35. The navigation system 20 may track these objects for purposes of determining their relative poses (i.e., positions and orientations) for use by the surgeon and, in some cases, for purposes of controlling or constraining movement of the surgical instrument 22 relative to virtual cutting boundaries (not shown) associated with the femur F and tibia T. An exemplary control scheme for the system 10 is shown in FIG. 3.

The navigation system 20 generally includes one or more computer cart assemblies 24 that houses one or more navigation controllers 26. A navigation interface is in operative communication with the navigation controller 26. The navigation interface includes one or more displays 28, 29 adjustably mounted to the computer cart assembly 24 or mounted to separate carts as shown. Input devices I such as a keyboard(s) and a mouse(s) may be used to input information into the navigation controller 26 or otherwise select/control certain aspects of the navigation controller 26. Other input devices I are contemplated including a touch screen, voice-activation, gesture sensors, and the like.

A surgical navigation localizer 34 communicates with the navigation controller 26. In the embodiment shown, the localizer 34 is an optical localizer and includes a camera unit 36. In other embodiments, the localizer 34 employs other modalities for tracking, e.g., radio frequency (RF), infrared (IR), ultrasonic, electromagnetic, inertial, and the like. The camera unit 36 has a housing 38 comprising an outer casing that houses one or more optical position sensors 40. In some embodiments at least two optical sensors 40 are employed, preferably three or four. The optical sensors 40 may be separate charge-coupled devices (CCD). In one embodiment three, one-dimensional CCDs are employed. Two-dimensional or three-dimensional sensors could also be employed. It should be appreciated that in other embodiments, separate camera units, each with a separate CCD, or two or more CCDs, could also be arranged around the operating room. The CCDs detect light signals, such as infrared (IR) signals.

The camera unit 36 may be mounted on an adjustable arm to position the optical sensors 40 with a field-of-view of the below discussed trackers that, ideally, is free from obstructions. In some embodiments the camera unit 36 is adjustable in at least one degree of freedom by rotating about a rotational joint. In other embodiments, the camera unit 36 is adjustable about two or more degrees of freedom.

The camera unit 36 includes a camera controller 42 in communication with the optical sensors 40 to receive signals from the optical sensors 40. The camera controller 42 communicates with the navigation controller 26 through either a wired or wireless connection (not shown). One such connection may be an IEEE 1394 interface, which is a serial bus interface standard for high-speed communications and isochronous real-time data transfer. The connection could also use a company specific protocol. In other embodiments, the optical sensors 40 communicate directly with the navigation controller 26.

Position and orientation signals and/or data are transmitted to the navigation controller 26 for purposes of tracking objects. The computer cart assembly 24, the display 28, and the camera unit 36 may be like those described in U.S. Pat. No. 7,725,162 to Malackowski, et al. issued on May 25, 2010, entitled “Surgery System,” hereby incorporated by reference.

The navigation controller 26 can be a personal computer or laptop computer. Navigation controller 26 has the displays 28, 29, central processing unit (CPU) and/or other processors, memory (not shown), and storage (not shown). The navigation controller 26 is loaded with software as described below. The software converts the signals received from the camera unit 36 into data representative of the position and orientation of the objects being tracked.

Navigation system 20 is operable with a plurality of tracking devices 44, 46, 48, also referred to herein as trackers. In the illustrated embodiment, one tracker 44 is firmly affixed to the femur F of the patient and another tracker 46 is firmly affixed to the tibia T of the patient. Trackers 44, 46 are firmly affixed to sections of bone. Trackers 44, 46 may be attached to the femur F and tibia T in the manner shown in U.S. Pat. No. 7,725,162, incorporated above. Trackers 44, 46 could also be mounted like those shown in U.S. Pat. Pub. No. 2014/0200621, entitled, “Navigation Systems and Methods for Indicating and Reducing Line-of-Sight Errors,” hereby incorporated by reference. In additional embodiments, a tracker (not shown) is attached to the patella to track a position and orientation of the patella. In yet further embodiments, the trackers 44, 46 could be mounted to other tissue types or parts of the anatomy.

One of the tracking devices 48 may be realized as an instrument tracker 48. In the illustrated embodiment, the instrument tracker 48 is shown coupled to the manipulator 12. Alternatively, the instrument tracker 48 may be integrated into the surgical instrument 22 during manufacture or may be separately mounted to the surgical instrument 22 (or to an end effector attached to the manipulator 12 of which the surgical instrument 22 forms a part) in preparation for surgical procedures. The working end of the surgical instrument 22, which is being tracked by virtue of the instrument tracker 48, may be referred to herein as an energy applicator, and may be a rotating bur, electrical ablation device, probe, or the like.

In the embodiment shown, the surgical instrument 22 is attached to the manipulator 12. Such an arrangement is shown in U.S. Pat. No. 9,119,655, entitled, “Surgical Manipulator Capable of Controlling a Surgical Instrument in Multiple Modes,”hereby incorporated by reference.

Generally, the surgical instrument 22 forms part of the end effector of the manipulator 12. The manipulator 12 has a base 57, a plurality of links 58 extending from the base 57, and a plurality of active joints (not numbered) for moving the surgical instrument 22 with respect to the base 57. The manipulator 12 has the ability to operate in a manual mode or a semi-autonomous mode in which the surgical instrument 22 is moved along a predefined instrument path, as described in U.S. Pat. No. 9,119,655, entitled, “Surgical Manipulator Capable of Controlling a Surgical Instrument in Multiple Modes,” incorporated above, or the manipulator 12 may be configured to move in the manner described in U.S. Pat. No. 8,010,180, hereby incorporated by reference.

The manipulator controller 54 may have a central processing unit (CPU) and/or other manipulator processors, memory (not shown), and storage (not shown). The manipulator controller 54, also referred to as a manipulator computer, is loaded with software as described below. The manipulator processors could include one or more processors to control operation of the manipulator 12. The processors can be any type of microprocessor or multi-processor system. The term processor is not intended to limit any embodiment to a single processor.

The manipulator may include a plurality of position sensors S associated with the plurality of links 58 of the manipulator 12. In one embodiment, the position sensors S are encoders. The position sensors S may be any suitable type of encoder, such as rotary encoders. Each position sensor S is associated with a joint actuator, such as a joint motor M. Each position sensor S is a sensor that monitors the angular position of one of six motor driven links 58 of the manipulator 12 with which the position sensor S is associated. Multiple position sensors S may be associated with each joint of the manipulator 12 in some embodiments. The manipulator 12 may be in the form of a conventional robot or other conventional machining apparatus, and thus the components thereof shall not be described in detail.

In order to determine the current location of the surgical instrument 22, data from the position sensors S may be used to determine measured joint angles. The measured joint angles of the joints are forwarded to a forward kinematics module, as known in the art. Based on the measured joint angles and preloaded data, the forward kinematics module determines the pose of the surgical instrument 22 in a manipulator coordinate system MNPL. The preloaded data are data that define the geometry of the plurality of links 58 and joints. With this encoder-based data, the manipulator controller 54 and/or navigation controller 26 can transform coordinates from the localizer coordinate system LCLZ into the manipulator coordinate system MNPL, vice versa, or can transform coordinates from one coordinate system into any other coordinate system described herein using conventional transformation techniques. In many cases, the coordinates of interest associated with the surgical instrument 22 (e.g., the tool center point or TCP), the virtual boundaries, and the tissue being treated, are transformed into a common coordinate system for purposes of relative tracking and display. An exemplary system for determining the current location of a surgical instrument is provided by U.S. Pat. Pub. No. 2020/0078100, entitled, “Systems and Methods for Surgical Navigation,” the entirety of which is hereby incorporated by reference.

In some modes, the manipulator controller 54 determines the desired location to which the surgical instrument 22 should be moved. Based on this determination, and information relating to the current location (e.g., pose) of the surgical instrument 22, the manipulator controller 54 determines the extent to which each of the plurality of links 58 needs to be moved in order to reposition the surgical instrument 22 from the current location to the desired location. The data regarding where the plurality of links 58 are to be positioned is forwarded to joint motor controllers JMCs that control the joints of the manipulator 12 to move the plurality of links 58 and thereby move the surgical instrument 22 from the current location to the desired location. In other modes, the manipulator 12 is capable of being manipulated as described in U.S. Pat. No. 8,010,180, hereby incorporated by reference, in which case the actuators are controlled by the manipulator controller 54 to provide gravity compensation to prevent the surgical instrument 22 from lowering due to gravity and/or to activate in response to a user attempting to place the working end of the surgical instrument 22 beyond a virtual boundary.

The optical sensors 40 of the localizer 34 may receive light signals from the trackers 44, 46, 48. In the illustrated embodiment, the trackers 44, 46, 48 are passive trackers. In this embodiment, each tracker 44, 46, 48 has at least three passive tracking elements or markers (e.g., reflectors) for transmitting light signals (e.g., reflecting light emitted from the camera unit 36) to the optical sensors 40. In other embodiments, active tracking markers can be employed. The active markers can be, for example, light emitting diodes transmitting light, such as infrared light. The navigation system may also be used with passive tracker arrangement.

Alternatively, the navigation system 20 may track the pose of real objects in the operating room via a marker-less tracking system. For example, the marker-less tracking system may determine the pose of the surgical instrument 22 as the instrument 22 is moved around the operating room. The marker-less tracking system may also determine poses of certain aspects of the patient. For example, the marker-less tracking system may estimate joint positions, anatomical coordinate systems, global coordinate systems, and other characteristics of the patient based on data collected by the navigation system 20. An exemplary marker-less tracking system is shown in U.S. Pat. No. 7,257,237, entitled, “Real Time Markerless Motion Tracking Using Linked Kinematic Chains,” the disclosure of which is hereby incorporated by reference.

The navigation controller 26 includes a navigation processor. It should be understood that the navigation processor could include one or more processors to control operation of the navigation controller 26. The processors can be any type of microprocessor or multi-processor system. The term processor is not intended to limit the scope of any embodiment to a single processor.

The camera unit 36 generally receives optical signals from the trackers 44, 46, 48 and outputs to the navigation controller 26 signals relating to the position of the tracking markers of the trackers 44, 46, 48 relative to the localizer 34. Based on the received optical signals, navigation controller 26 generates data indicating the relative positions and orientations of the trackers 44, 46, 48 relative to the localizer 34. In one version, the navigation controller 26 uses well known triangulation methods for determining position data.

Prior to the start of the surgical procedure, additional data may be loaded into the navigation controller 26. Based on the position and orientation of the trackers 44, 46, 48 and the previously loaded data, the navigation controller 26 determines the position of the working end of the surgical instrument 22 (e.g., the centroid of a surgical bur) and/or the orientation of the surgical instrument 22 relative to the tissue against which the working end is to be applied. In some embodiments, the navigation controller 26 forwards these data to a manipulator controller 54. The manipulator controller 54 can then use the data to control the manipulator 12. This control can be like that described in U.S. Pat. No. 9,119,655, entitled, “Surgical Manipulator Capable of Controlling a Surgical Instrument in Multiple Modes,” incorporated above, or like that described in U.S. Pat. No. 8,010,180, entitled, “Haptic Guidance System and Method,” also incorporated above.

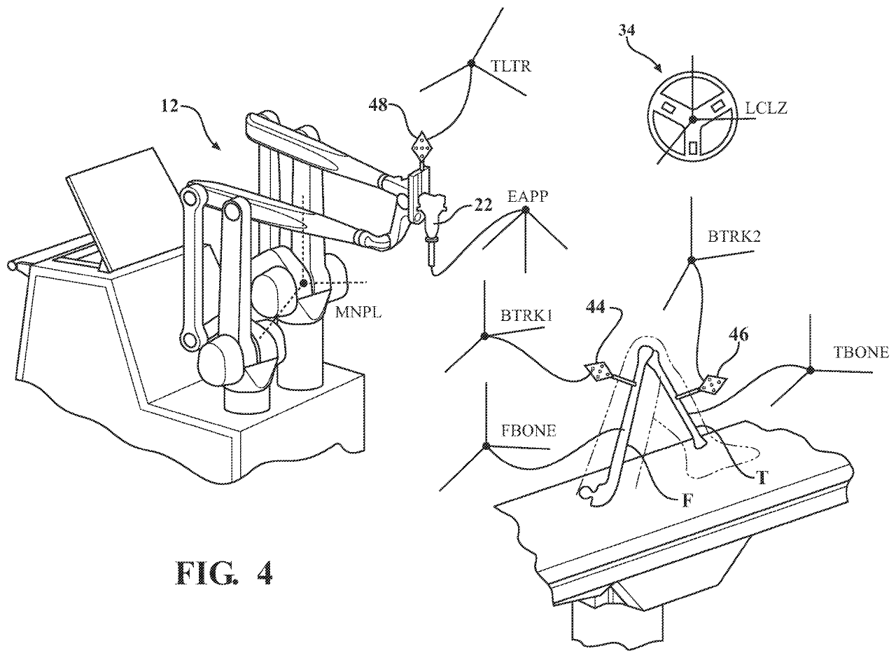

Referring to FIG. 4, the tracking of objects is generally conducted with reference to a localizer coordinate system LCLZ. The localizer coordinate system has an origin and an orientation (a set of x, y, and z axes). During the procedure one goal is to keep the localizer coordinate system LCLZ in a known position. An accelerometer (not shown) mounted to the localizer 34 may be used to track sudden or unexpected movement of the localizer coordinate system LCLZ, as may occur when the localizer 34 is inadvertently bumped by surgical personnel.

Each tracker 44, 46, 48 and object being tracked also has its own coordinate system separate from the localizer coordinate system LCLZ. Components of the navigation system 20 that have their own coordinate systems are the bone trackers 44, 46 (only one of which is shown in FIG. 4) and the instrument tracker 48. These coordinate systems are represented as, respectively, bone tracker coordinate systems BTRK1, BTRK2, and instrument tracker coordinate system TLTR.

The navigation system 20 monitors the positions of the femur F and tibia T of the patient by monitoring the position of bone trackers 44, 46 firmly attached to bone. Femur coordinate system is FBONE and tibia coordinate system is TBONE, which are the coordinate systems of the bones to which the bone trackers 44, 46 are firmly attached. The femur and tibia coordinate systems FBONE, TBONE may be aligned with the anatomical coordinate system of the respective bone, or the mechanical coordinate system associated with the respective bone. Alternatively, the anatomical coordinate system of each bone could be otherwise associated with the respective coordinate systems FBONE, TBONE. Herein, “anatomical coordinate system” may be understood to refer to the femur and/or tibia coordinate system FBONE, TBONE, an anatomical coordinate system associated with one of the femur F and tibia T, an anatomical coordinate system associated with both the femur F and the tibia T, an anatomical coordinate system aligned with the mechanical axis of the femur and/or tibia (i.e. with the joints associated therewith), or any other coordinate system sharing an association with at least one of the femur F and the tibia T.

Prior to the start of the procedure, pre-operative images of the femur F and tibia T may be generated (or of other tissues in other embodiments). These images may be based on MRI scans, radiological scans or computed tomography (CT) scans of the patient's anatomy. These images or three-dimensional models developed from these images, may be mapped to the femur coordinate system FBONE and tibia coordinate system TBONE using well known methods in the art (see transform T11). These images/models are fixed in the femur coordinate system FBONE and tibia coordinate system TBONE. As an alternative to taking pre-operative images, plans for treatment can be developed in the operating room (OR) from kinematic studies, bone tracing, and other methods. The models described herein may be represented by mesh surfaces, constructive solid geometry (CSG), voxels, or using other model constructs.

In an exemplary method, during an initial phase of the procedure, the bone trackers 44, 46 are firmly affixed to the bones of the patient. The pose (position and orientation) of coordinate systems FBONE and TBONE are mapped to coordinate systems BTRK1 and BTRK2, respectively. In one embodiment, a pointer instrument, such as disclosed in U.S. Pat. No. 7,725,162 to Malackowski, et al., incorporated above, having its own tracker, may be used to register the femur coordinate system FBONE and tibia coordinate system TBONE to the bone tracker coordinate systems BTRK1 and BTRK2, respectively, and in some cases, also provides input for mapping the models of the femur F and tibia to the femur coordinate system FBONE and the tibia coordinate system TBONE (e.g., by touching anatomical landmarks on the actual bone that are also identified in the models so that the models can be fit to the bone using known best-fit matching techniques). Given the fixed relationship between the bones and their bone trackers 44, 46, positions and orientations of the femur F and tibia T in the femur coordinate system FBONE and tibia coordinate system TBONE can be transformed to the bone tracker coordinate systems BTRK1 and BTRK2 so the camera unit 36 is able to track the femur F and tibia T by tracking the bone trackers 44, 46. These pose-describing data are stored in memory integral with both manipulator controller 54 and navigation controller 26. One exemplary method is provided by U.S. Pat. No. 11,369,438, entitled, “Navigation Systems and Methods for Indicating and Reducing Line-Of-Sight Errors,” which is hereby incorporated by reference.

The working end of the surgical instrument 22 has its own coordinate system. In some embodiments, the surgical instrument 22 comprises a handpiece and an accessory that is removably coupled to the handpiece. The accessory may be referred to as the energy applicator and may comprise a bur, an electrosurgical tip, an ultrasonic tip, or the like. Thus, the working end of the surgical instrument 22 may comprise the energy applicator. The coordinate system of the surgical instrument 22 is referenced herein as coordinate system EAPP. The origin of the coordinate system EAPP may represent a centroid of a surgical cutting bur, for example. In other embodiments, the accessory may simply comprise a probe or other surgical instrument with the origin of the coordinate system EAPP being a tip of the probe. The pose of coordinate system EAPP is registered to the pose of instrument tracker coordinate system TLTR before the procedure begins (see transforms T1, T2, T3). Accordingly, the poses of these coordinate systems EAPP, TLTR relative to each other are determined. The pose-describing data are stored in memory integral with both manipulator controller 54 and navigation controller 26.

Referring back to FIG. 2, in some embodiments, the navigation system 20 includes a localization engine 100. Components of the localization engine 100 run on navigation controller 26. In some embodiments, the localization engine 100 may run on the manipulator controller 54. Localization engine 100 receives as inputs the optically-based signals from the camera controller 42 and, in some embodiments, non-optically based signals from the tracker controller. Based on these signals, localization engine 100 determines the pose of the bone tracker coordinate systems BTRK1 and BTRK2 in the localizer coordinate system LCLZ. Based on the same signals received for the instrument tracker 48, the localization engine 100 determines the pose of the instrument tracker coordinate system TLTR in the localizer coordinate system LCLZ.

The localization engine 100 forwards the signals representative of the poses of trackers 44, 46, 48 to a coordinate transformer 101. The coordinate transformer 101 is a navigation system software module that runs on navigation controller 26. The coordinate transformer 101 references the data that defines the relationship between the pre-operative images of the patient and the bone trackers 44, 46. Coordinate transformer 101 also stores the data indicating the pose of the working end of the surgical instrument 22 relative to the instrument tracker 48.

During the procedure, the coordinate transformer 101 receives the data indicating the relative poses of the trackers 44, 46, 48 to the localizer 34. Based on these data, the previously loaded data, and encoder data from the manipulator 12, the coordinate transformer 101 generates data indicating the relative positions and orientations of the coordinate system EAPP and the bone coordinate systems, FBONE and TBONE.

As a result, coordinate transformer 101 generates data indicating the position and orientation of the working end of the surgical instrument 22 relative to the tissue (e.g., bone) against which the working end is applied. Image signals representative of these data are forwarded to displays 28, 29 enabling the surgeon and staff to view this information. In certain embodiments, other signals representative of these data can be forwarded to the manipulator controller 54 to guide the manipulator 12 and corresponding movement of the surgical instrument 22.

Coordinate transformer 101 is also operable to determine the position and orientation (pose) of any coordinate system described herein relative to another coordinate system by utilizing known transformation techniques, e.g., translation and rotation of one coordinate system to another based on various transforms described herein. As is known, the relationship between two coordinate systems is represented by a six degree of freedom relative pose, a translation followed by a rotation, e.g., the pose of a first coordinate system in a second coordinate system is given by the translation from the second coordinate system's origin to the first coordinate system's origin and the rotation of the first coordinate system's coordinate axes in the second coordinate system. The translation is given as a vector. The rotation is given by a rotation matrix.

An exemplary system and method for determining the pose of each of the tracker coordinate systems BTRK1, BTRK2, TLTR in the localizer coordinate system LCLZ and systems and methods for determining the pose of the trackers 44, 46, 48 and the corresponding poses of the surgical instrument 22 with respect to the femur F and tibia T are described in greater detail in U.S. Pat. No. 9,008,757, entitled “Navigation System Including Optical and Non-Optical Sensors”, hereby incorporated by reference.

An exemplary system and method for transforming the coordinate systems associated with the instrument 22, the patient, the patient's anatomy (e.g. FBONE, TBONE), other aspects of the system 20, and one or more of the planning parameters registered by the navigation system 20 into another one of the coordinate systems provided herein is described in greater detail in U.S. Pat. Pub. No. 2022/0151703, entitled, “Patient-Specific Preoperative Planning Simulation Techniques,” hereby incorporated by reference.

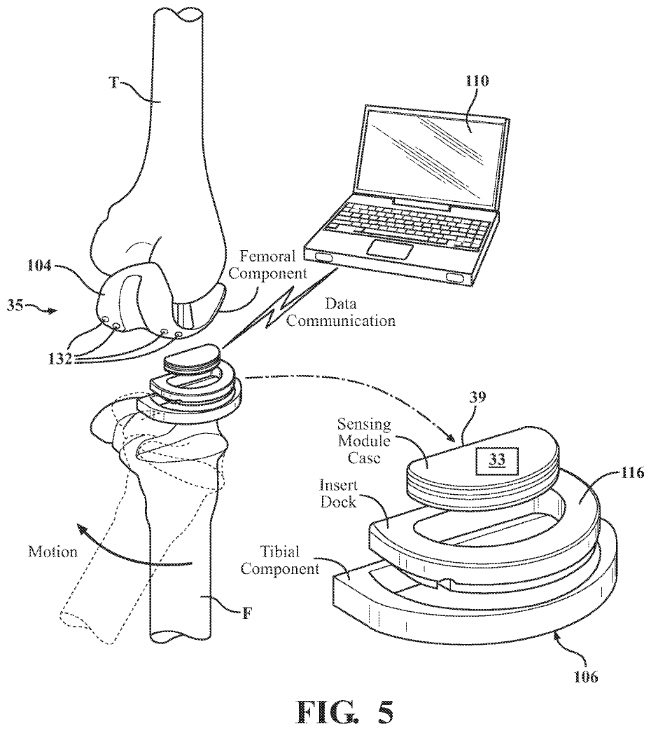

Referring to FIG. 5, after desired volumes from the tibia T and the femur F have been removed, the knee implant 35 may be installed. As discussed previously, the implant 35 may include a femoral component 104 and a tibial component 106. The tibial component 106 may include an implant sensing module 33 for measuring a parameter in accordance with an exemplary embodiment and the femoral component 104 may include a plurality of magnets 132. The parameter may correspond to acceleration or rotational motion which can occur when the knee implant and thus the sensing module 33 is moved or put in motion.

The implant sensing module 33 may also include a hall effect sensor 62 and electronics circuitry. The electronic circuitry manages and controls various operations of the components of the sensing module 33, such as sensing, power management, telemetry, and acceleration sensing. It can include analog circuits, digital circuits, integrated circuits, discrete components, or any combination thereof. In one arrangement, it can be partitioned among integrated circuits and discrete components to minimize power consumption without compromising performance. Partitioning functions between digital and analog circuit enhances design flexibility and facilitates minimizing power consumption without sacrificing functionality or performance. Accordingly, the electronic circuitry can comprise one or more Application Specific Integrated Circuit (ASIC) chips, for example, specific to a core signal processing algorithm.

In another arrangement, the electronic circuitry can comprise a controller such as a programmable processor, a Digital Signal Processor (DSP), a microcontroller, or a microprocessor, with associated storage memory and logic. The controller can utilize computing technologies with associated storage memory such a Flash, ROM, RAM, SRAM, DRAM or other like technologies for controlling operations of the aforementioned components of the sensing module. In one arrangement, the storage memory may store one or more sets of instructions (e.g., software) embodying any one or more of the methodologies or functions described herein. The instructions may also reside, completely or at least partially, within other memory, and/or a processor during execution thereof by another processor or computer system.

The electronics assemblage also supports testability and calibration features that assure the quality, accuracy, and reliability of the completed wireless sensing module or device. A temporary bi-directional interconnect assures a high level of electrical observability and controllability of the electronics. The test interconnect also provides a high level of electrical observability of the sensing subsystem, including the transducers, waveguides, and mechanical spring or elastic assembly. Carriers or fixtures emulate the final enclosure of the completed wireless sensing module or device during manufacturing processing thus enabling capture of accurate calibration data for the calibrated parameters of the finished wireless sensing module or device. These calibration parameters are stored within the on-board memory integrated into the electronics assemblage.

Applications for implant sensing module 33 may include, but are not limited to, disposable modules or devices as well as reusable modules or devices and modules or devices for long-term use. In addition to non-medical applications, examples of a wide range of potential medical applications may include, but are not limited to, implantable devices, modules within implantable devices, intra-operative implants or modules within intra-operative implants or trial inserts, modules within inserted or ingested devices, modules within wearable devices, modules within handheld devices, modules within instruments, appliances, equipment, or accessories of all of these, or disposables within implants, trial inserts, inserted or ingested devices, wearable devices, handheld devices, instruments, appliances, equipment, or accessories to these devices, instruments, appliances, or equipment.

The plurality of magnets 132 of the femoral component 104 cause the hall-effect sensor 62 to generate a signal which may be used to derive a reference pose of the femur F so that an exact angular relationship between the femur F and tibia T may be determined.

The implant including the femoral component 104 and the tibial component 106 may include various features discussed in U.S. Pat. No. 9,125,627, entitled, “Wireless power modulation telemetry for measuring a parameter of the muscular-skeletal system,” and U.S. Provisional Patent Application No. 63/309,809, entitled, “Implant Encoder,” both of which are hereby incorporated by reference.

Generally, a proximal end of tibia Fis prepared to receive tibial prosthetic component 106. The tibial component 106 is a support structure that is fastened to the proximal end of the tibia and is usually made of a metal or metal alloy. The tibial prosthetic component 106 also retains the insert in a fixed position with respect to tibia F. Similarly, a distal end of femur T is prepared to receive femoral prosthetic component 104. The femoral prosthetic component 104 is generally shaped to have an outer condylar articulating surface. The preparation of femur T and tibia F is aligned to the mechanical axis of the leg. The implant sensing module 33 provides a concave or flat surface against which the outer condylar articulating surface of the femoral prosthetic component 104 rides relative to the tibia prosthetic component 106. In particular, the top surface of the implant sensing module 33 faces the condylar articulating surface of the femoral prosthetic component 104, and the bottom surface of an insert dock 116 faces the top surface of the tibial prosthetic component 106.

The implant 35 emulates the function of a natural knee joint. The implant sensing module 33 can measure loads or other parameters at various points throughout the range of motion. Data from the implant sensing module 33 may be transmitted to the navigation controller 26 and/or a receiving station 110 utilizing any sort of wireless communication protocol. While the implant sensing module 33 is contemplated at a permanent component, the implant sensing module 33 may be removed at any time and replaced with a substitute implant sensing module 33 if any of the components were to stop functioning. In another implementation, the implant sensing module 33 may be a permanent component of the replacement joint. The implant sensing module 33 may be used to provide both short term and long term post-operative data on the implanted joint. The implant sensing module 33 may also be coupled proximal to the implant or joint and directly to the muscular-skeletal system. In all of the embodiments, receiving station 110 can include data processing, storage, or display, or combination thereof and provide real time graphics of various data provided by the implant sensing module 33 such as range of motion for the joint while performing any sort of movement or exercise. The receiving station may generate one or more graphical user interfaces as described in U.S. Pat. No. 11,337,649 entitled “Systems and Methods for monitoring physical therapy of the knee and other joints,” hereby incorporated by reference.

When the implant 35 is coupled to the leg, the sensor coordinate system of an IMU 64 coupled to the implant 35 may be offset with one or more axes or lines (e.g., the mechanical axis, the anatomical axis, the joint line) of the patient. For example, the sensor coordinate system of the IMU 64 coupled to tibial component 106 via the final insert 118 may be offset by the mechanical axis of the tibia T or the anatomical axis of the tibia T.

The implant 35 and its associated components may be like the implant provided by U.S. Pat. No. 9,125,627, entitled, “Wireless power modulation telemetry for measuring a parameter of the muscular-skeletal system,”hereby incorporated by reference.

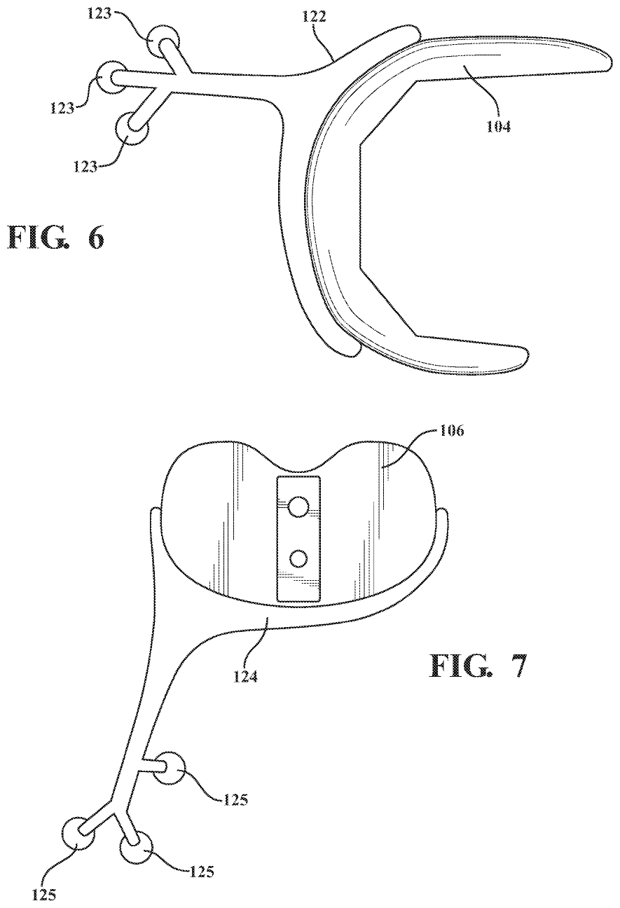

Referring to FIGS. 6 and 7, a femoral implant attachment 122 and a tibial implant attachment 124 are shown. The femoral implant attachment 122 is shown coupled to the femoral component 104 in FIG. 6, and the tibial implant attachment 124 is shown coupled to the tibial component 106 in FIG. 7. Both of these couplings are generally removable couplings such that the implant attachments 122, 124 can be detached from the femoral component 104 and the tibial component 106, respectively. The implant attachments 122, 124 may be removably coupled to the components 104, 106 in any suitable manner. For example, the implant attachments 122, 124 may be removably coupled to the components 104, 106 via magnets, fasteners, adhesives, and/or corresponding geometry such that a face of one of the implant attachments 122, 124 or components 104, 106 is shaped to receive a face of the other of the implant attachments 122, 124 or components 104, 106.

The femoral implant attachment 122 and the tibial implant attachment 124 each generally include a series of trackers 123, 125. The trackers 123, 125 allow the navigation system 20 to detect the pose of the implant attachments 122, 124 based on a known relationship between the trackers 123, 125 and the rest of the geometry of the respective implant attachment 122, 124. In some embodiments, the implant attachments 122, 124 may be configured to be removably couplable to the components 104, 106 according to a predefined relationship. In other words, the attachments 122, 124 may be configured to be removably coupled to the components 104, 106 in such a way that the relationship between the pose of the attachments 122, 124 and the pose of the respective components 104, 106 is known to the navigation controller 26. For example, the navigation controller 26 may know the relationship between the pose of the femoral implant attachment 122 and the pose of the femoral component 104 when the femoral implant attachment 122 is removably coupled to the femoral component 104 based on the predefined relationship. As such, the navigation controller 26 may be configured to determine the pose of the femoral component 104 (which may not include trackers), based on a combination of the detected pose of the femoral implant attachment 122 and the predefined relationship between the two 104, 122. In a similar manner, the navigation controller 26 may be configured to determine the pose of the tibial component 106 (which may not include trackers), based on a combination of the detected pose of the tibial implant attachment 124 and the predefined relationship between the two 106, 124. This process is described in more detail below.

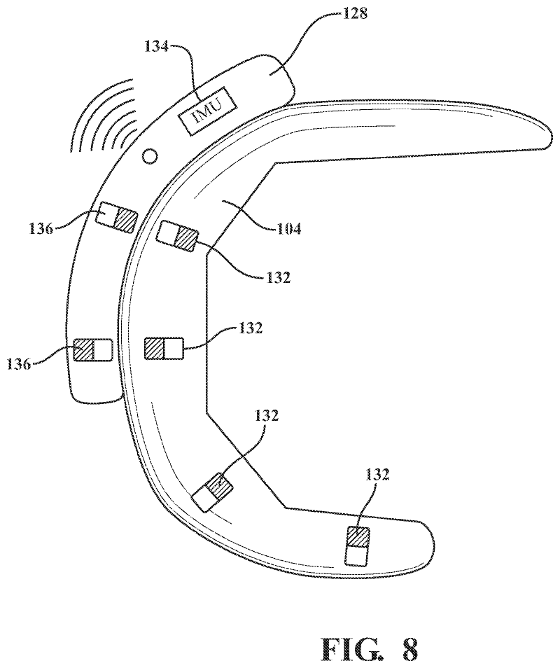

Referring to FIG. 8, a femoral IMU attachment 128 is shown coupled to the femoral component 104 via magnets 132 included in the femoral component 104 and corresponding magnets 136 included in the femoral IMU attachment 128. The femoral IMU attachment 128 may be shaped to couple to the femoral component 104 such that the pose of the femoral component 104 is known relative to the pose of the femoral IMU attachment 128 according to a predefined relationship known to the navigation controller 26. The magnets 132, 136 may be disposed at certain locations on the femoral component 104 and femoral IMU attachment 128 in order to facilitate a predefined coupling location. Further, although the illustrated embodiment shows the magnets 132, 136 in various positions, the magnets 132, 136 may be disposed anywhere in/on the femoral component 104 and femoral IMU attachment 128, respectively.

The femoral IMU attachment 128 may include communication means such that the femoral IMU attachment 128 is capable of communicating with the navigation system 20. Further, the femoral IMU attachment 128 may include an auxiliary IMU 134 similar to the IMU 64. As such, the femoral IMU attachment 128 may transmit data to the navigation system 20 corresponding to the pose of the femoral IMU attachment 128 according to the auxiliary IMU 134. As will be described in more detail below, the navigation controller 26 may be configured to determine the pose of the sensor coordinate system based on the pose of the auxiliary IMU coordinate system and the relationship between the pose of the femoral component 104 and the pose of the femoral IMU attachment 128.

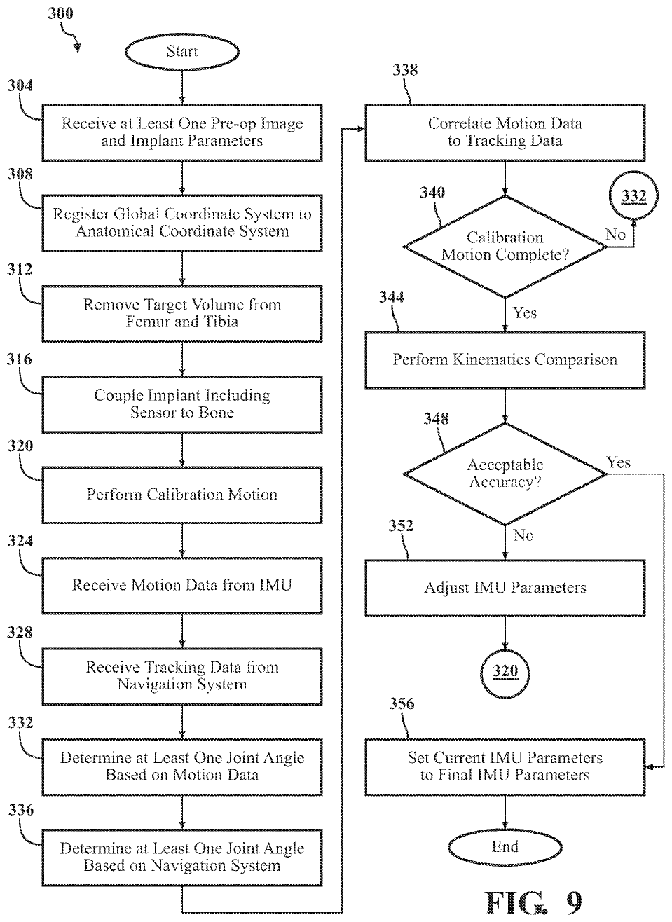

Referring to FIGS. 9 to 15, exemplary methods according to the present disclosure are depicted. The exemplary methods aim to register the sensor coordinate system to the anatomical coordinate system in various ways using a combination of implanted IMU data, external IMU data, motion capture data, planning data, pre-op imaging data, intra-op imaging data, and post-op imaging data. The various types of data may be collected as described herein or may be collected in other manners. The phrase “is known” as used herein may mean that a component of the system 10 is capable of determining the element/data that is stated as “known.” For example, the phrase “data C is known to the navigation controller 26 based on data A and data B” may mean that data C is calculable by the navigation controller 26 via a mathematical (or alternative) combination of data A and data B. As will be appreciated from the subsequent description below, these methods merely represent exemplary and non-limiting sequence of blocks to describe the exemplary methods. While each of the exemplary methods are shown as “starting” and “ending” in FIGS. 9-15 for illustrative purposes, it will be appreciated that the methods may instead return to start.

As used herein, the terms “registration” and “register” are used to describe the process in which at least one of the axes of one coordinate system are brought into alignment with at least one of the axes of another coordinate system. For example, registering the sensor coordinate system of the IMU 64 to the anatomical coordinate system may include aligning at least one of the x-axis, y-axis, and z-axis of the sensor coordinate system with the corresponding x-axis, y-axis, and z-axis of the anatomical coordinate system. Further, registering the sensor coordinate system to the anatomical coordinate system may include aligned each of the x-axis, y-axis, and z-axis of the sensor coordinate system with the corresponding x-axis, y-axis, and z-axis of the anatomical coordinate system.

The below methods generally include step in which the parameters of the IMU 64 are adjusted such that the sensor readings from the IMU 64 are output relative to the anatomical coordinate system. It will be appreciated that this adjustment may be made by different components of the system 10. For example, the raw sensor data from the IMU 64 may be interpreted and altered by the IMU 64 prior to communicating with the rest of the navigation system 20. Alternatively, the raw sensor data from the IMU 64 may be output and read by the navigation controller 26, and the navigation controller 26 may include a parameter adjustment metric which is combined with the raw sensor data in order to understand the raw sensor data relative to the anatomical coordinate system instead of the sensor coordinate system. It is further contemplated to have any one or combination of the components of the system transform the raw sensor data from the IMU 64 into the anatomical coordinate system.

With reference to FIG. 9, a first exemplary method 300 according to the teachings of the present disclosure is depicted. At 304, the method 300 may receive at least one pre-op medical image depicting the femur F and the tibia T and one or more models of the femur F and or tibia T. At 308, the navigation controller 26 may register the at least one pre-op medical image and/or model of the femur F and/or the tibia T by registering the global coordinate system to the anatomical coordinate system of the patient.

The global coordinate system can be registered to the anatomical coordinate system by any suitable method. For example, the localizer 34 may define an image coordinate system corresponding to patient image data acquired by the camera unit 36, and the global coordinate system may be arbitrarily defined, defined by the operating room, or otherwise defined in a way known to the navigation controller 26. In such an example, the image coordinate system may have a relationship to the global coordinate system known by the navigation controller 26. As such, the medical image provides information to the navigation controller 26 corresponding to the pose of the femur F and the tibia T in the image coordinate system. Because of the known relationship between the image and global coordinate systems, the pose of the femur F and the tibia T are also known in the global coordinate system. Further, the anatomical coordinate system is defined by the femur F and the tibia T, and the pose of the anatomical coordinate system can be determined based on the pose of the femur F and the tibia T in the medical image. Therefore, the pose of the anatomical coordinate system can be known relative to the pose of the image and global coordinate systems, and the global coordinate system can be registered to the anatomical coordinate system of the patient.

At 312, the manipulator 12 and/or surgeon may remove a target volume of the tibia T and a target volume of the femur F according to the surgical plan. At 316, the manipulator 12 and/or surgeon may couple the implant 35 including the femoral component 106 and the tibial component 104 to the femur F and the tibia T, respectively. At 320, the surgeon may guide the leg of the patient through a calibration motion. At 324, the navigation controller 26 may receive motion data from the IMU 64 corresponding to the acceleration and rotation of the IMU 64 as detected by the sensors included in the IMU 64. The IMU 64 may calculate linear motion and angular orientation of the body part to which it is attached based on said acceleration and rotation (herein, “motion data”). For example, the IMU 64 may be included in the tibial component 106 and provide motion data pertaining to the tibia T to which the tibial component 106 is attached as detected by the IMU 64 sensors.

At 328, the navigation controller 26 may receive tracking data from the camera controller 42. For example, the navigation controller 26 may receive the pose of the trackers 44, 46 which are indicative of the pose of the femur F and the tibia T, respectively. At 332, the navigation controller 26 may determine at least one joint angle based on the motion data from the IMU 64 using an algorithm. At 336, the navigation controller 26 may determine at least one joint angle based on the tracking data from the camera controller 42. For example, the navigation controller 26 may use the relative pose of the femur F and the tibia T as indicated by the bone trackers 44, 46 to derive at least one joint angle. In another example, the navigation controller 26 may derive at least one joint angle from video analysis identifying joint centers in subsequent frames (optical or x-ray based). At 338, the navigation controller 26 may correlate the motion data to the tracking data.

At 340, the navigation controller 26 determines whether the calibration motion is complete 340. If so, the method 300 may continue at 344; otherwise, the method may continue at 332.

At 344, the navigation computer 26 may perform a kinematics comparison of the joint angle determined based on the motion data from the IMU 64 and the joint angle determined based on the navigation data from the navigation controller 26. At 348, the navigation controller 26 may determine whether the difference between the joint angle determined based on the motion data and the joint angle determine based on the navigation data is within a threshold range of accuracy of each other. For example, the threshold range of accuracy could be a number of degrees difference between the joint angle as determined based on the motion data and the joint angle as determined based on the navigation data. If so, the method 300 may continue at 356; otherwise, the method 300 may continue at 352. At 352, the method 300 may adjust one or more parameters of the IMU 64 stored on the navigation computer 26. For example, the navigation controller 26 may determine a relationship between ones of the axes or lines associated with the joint, tibia T, or femur D and at least one axis (i.e., the x-axis, the y-axis, or the z-axis) of the coordinate system of the IMU 64. The relationship may be an offset of the at least one axis relative to the at least one anatomical axis determined based on the navigation data.

After the navigation computer 26 adjusts the IMU parameters, the method 300 may continue back at 320 where the calibration motion is performed again. At 356, the method 300 sets the current parameters of the IMU 64 as the final parameters of the IMU 64 to register the coordinate system of the IMU to the anatomical coordinate system and the method 300 may end. The final parameters of the IMU 64 may correspond to a combination of the sensor coordinate system and the relationship between the sensor coordinate system and the anatomical coordinate system. As a result, the sensor coordinate system may be registered to the anatomical coordinate system such that sensor readings provided by the IMU 64 (e.g. translation and/or rotation of the sensor) are provided relative to the anatomical coordinate system instead of or in addition to the sensor coordinate system.

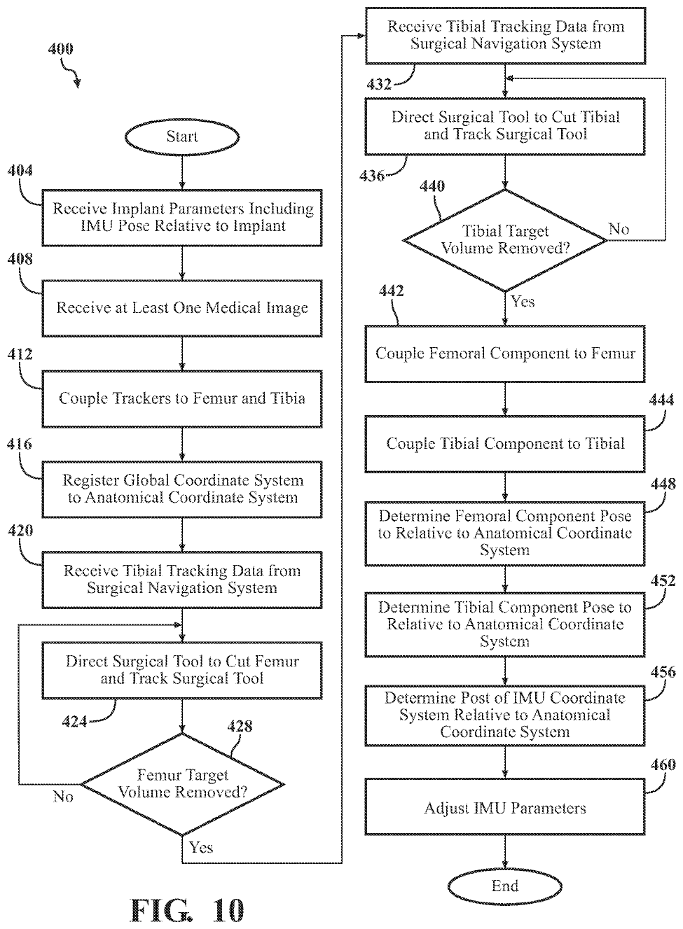

With reference to FIG. 10, a second exemplary method 400 according to the teachings of the present disclosure is depicted. At 404, the navigation controller 26 may receive implant parameters including the physical dimensions and geometrical profile of the implant 35 including the tibial component 106 and the femoral component 104 and pose of the IMU 64 relative to the tibial component 106 and/or the femoral component 104. For example, the navigation controller 26 may receive the physical dimensions and geometrical profile of the tibial component 106, as well as the pose of the sensor coordinate system relative to said physical dimensions and geometrical profile of the tibial component 106. At 408, the navigation controller 26 may receive at least one medical image of the femur F and the tibia T. At 412, the surgeon may couple the trackers 44, 46 to the femur F and the tibia T, respectively. At 416, the navigation controller 26 may register the global coordinate system as perceived by the localizer 34 to the anatomical coordinate system of the patient.

At 420, the navigation controller 26 may receive femur tracking data which may be used to determine the pose of the femur F. At 424, the navigation controller 26 may direct the manipulator 12 and/or surgeon to cut the femur F according to the surgical plan and track the manipulator 12 and/or surgical instrument 22 while the femur F is being cut. At 428, the navigation computer 26 may determine whether the femur F target volume has been removed. If so, the method 400 may continue at 436; otherwise, the method 400 may continue at 432. At 432, the navigation controller 26 may receive tibial tracking data. At 436, the navigation controller 26 may direct the manipulator 12 and/or surgeon to cut the tibia T according to the surgical plan and track the manipulator 12 and/or surgical instrument 22 while the tibia T is being cut. At 440, the navigation controller 26 may determine whether the tibial target volume has been removed. If so, the method 400 may continue at 442; otherwise, the method 400 may continue at 436.

At 442, the manipulator 12 and/or surgeon may couple the femoral component 104 to the femur. During 442, the navigation processor 26 tracks the manipulator 12 and/or the surgical instrument 22 as well as the pose of the femoral component 104 relative to the pose of the manipulator or surgical instrument 12, 22. At 444, the manipulator 12 and/or surgeon may couple the tibial component 106 to the tibial T During 444, the navigation processor 26 tracks the manipulator 12 and/or the surgical instrument 22 as well as the pose of the tibial component 106 relative to the pose of the manipulator or surgical instrument 12, 22. At 448, the navigation controller 26 may determine the pose of the femoral component 104 relative to the anatomical coordinate system based on the pose of the manipulator or instrument 12, 22 as the femoral component 104 is coupled to the femur F. At 452, the navigation computer 26 may determine the pose of the tibial component 106 relative to the anatomical coordinate system based on the pose of the manipulator or instrument 12, 22 as the tibial component 106 is coupled to the tibia T.

During both 448 and 452, the relationship between the pose of the component 104, 106 and the pose of the manipulator/instrument 12, 22 being used to couple the component to the femur F or tibia T is known by the navigation controller 26. Further, the pose of the manipulator/instrument 12, 22 is known relative to the anatomical coordinate system since the pose of the manipulator/instrument 12, 22, as well as the pose of the femur F and tibia T, are known relative to the global coordinate system. Because the global coordinate system was registered to the anatomical coordinate system at 416, the pose of the manipulator/instrument 12, 22 is known relative to the anatomical coordinate system as at least one of the components 104, 106 are coupled to the respective bone.

At 456, the navigation computer 26 may determine the pose of the sensor coordinate system of the IMU 64 relative to the anatomical coordinate system. More specifically, since the pose of the sensor coordinate system is known relative to the physical parameters and geometrical profile of the implant, and the pose of the implant relative to the anatomical coordinate system is also known, the pose of the sensor coordinate system can be registered to the anatomical coordinate system. At 460, the navigation computer 26 may adjust the parameters of the IMU 64 such that the sensor readings from the IMU 64 are output relative to the anatomical coordinate system and the method 400 may then end.

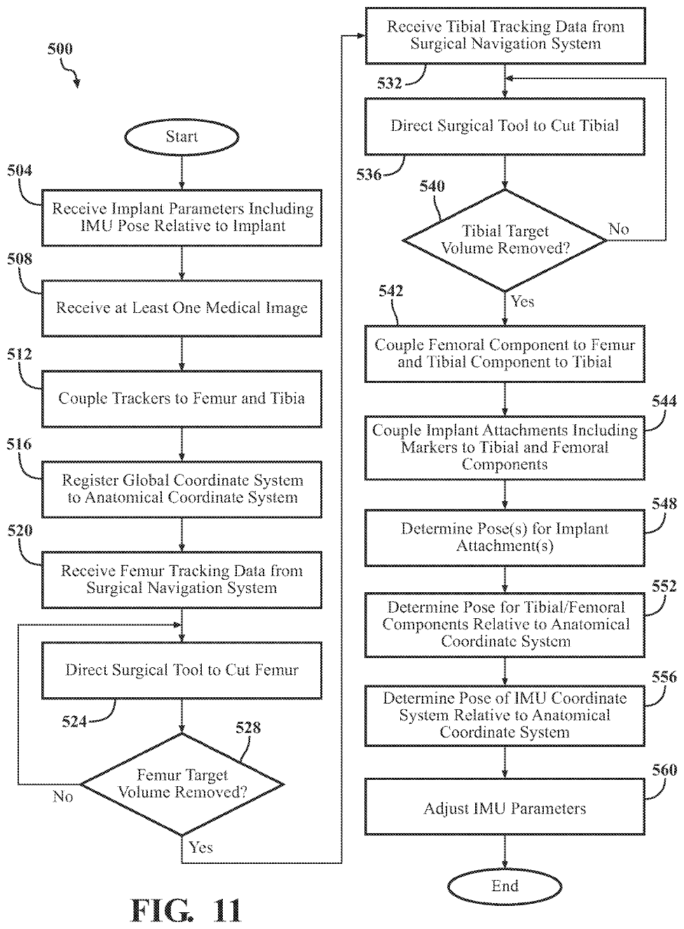

With reference to FIG. 11, a third exemplary method 500 according to the teachings of the present disclosure is depicted. The third exemplary method 500 is similar to the second exemplary method 400, except that the navigation controller 26 determines the pose of the components 104, 106 in the global coordinate system based on the pose of trackers coupled to the components 104, 106 after the components 104, 106 are fixed to the patient, as opposed to based on the pose of the manipulator/instrument 12, 22 as the components 104, 106 are fixed to the patient. At 504, the navigation controller 26 may receive implant parameters including the physical dimensions and geometrical profile of the implant 35 including the tibial component 106 and the femoral component 104 and pose of the IMU 64 relative to one of the tibial component 106. At 508, the navigation controller 26 may receive at least one medical image of the femur F and the tibia T. At 512, the surgeon may couple the tracker 44, 46 to the femur F and the tibia T, respectively. At 516, the navigation controller 26 may register the global coordinate system to the anatomical coordinate system.

At 520, the navigation controller 26 may receive femur tracking data which may be used to determine a pose of the femur F and thus the anatomical axis of the femur F. At 524, the navigation controller 26 may direct the manipulator 12 and/or surgeon to cut the femur F according to the surgical plan and track the manipulator 12 and/or surgical instrument 22 while the femur F is being cut. At 528, the navigation computer 26 may determine whether the femur F target volume has been removed. If so, the method 500 may continue at 532; otherwise, the method 500 may continue back at 524. At 532, the navigation controller 26 may receive tibial tracking data which may be used to determine a pose of the tibia T and thus the anatomical axis of the tibia T. At 536, the navigation controller 26 may direct the manipulator 12 and/or surgeon to cut the tibia T according to the surgical plan and track the manipulator 12 and/or surgical instrument 22 while the tibia T is being cut. At 540, the navigation controller 26 may determine whether the tibial target volume has been removed. If so, the method 500 may continue at 542; otherwise, the method 500 may continue at 436.

At 542, the manipulator 12 and/or surgeon may couple the femoral component 104 to the femur and couple the tibial component 106 to the tibia T. At 544, the femoral implant attachment 122 may be removably coupled to the femoral component 104, and the tibial implant attachment 124 may be removably coupled to the tibial component 106. At 548, the navigation controller 26 may determine a pose for the femoral implant attachment 122 and a pose for the tibial implant attachment 124. At 552, the navigation controller 26 may determine a pose of the tibial component 106 based on a pose of the tibial implant attachment 124 and a pose of the femoral component 104 based on a pose of the femoral implant attachment 122. Both of the pose of the tibial component 106 and the pose of the femoral component 104 are determined relative to the anatomical coordinate system of the patient.

At 556, the navigation controller 26 may determine a pose of the sensor coordinate system relative to the anatomical coordinate system based on the pose of the tibial component 106. More specifically, since the pose of the sensor coordinate system is known relative to the physical parameters and geometrical profile of the tibial component 106, and the pose of the femoral component 106 relative to the anatomical coordinate system is also known, the pose of the sensor coordinate system can be registered to the anatomical coordinate system. At 560, the navigation controller 26 may adjust the parameters of the IMU 64 such that the sensor readings from the IMU 64 are output relative to the anatomical coordinate system and the method 500 may end.

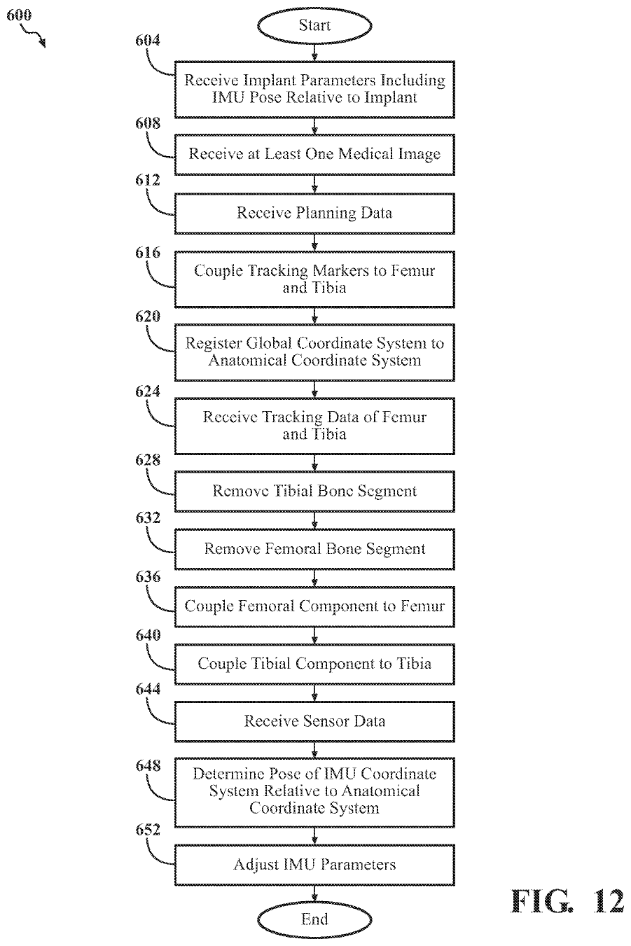

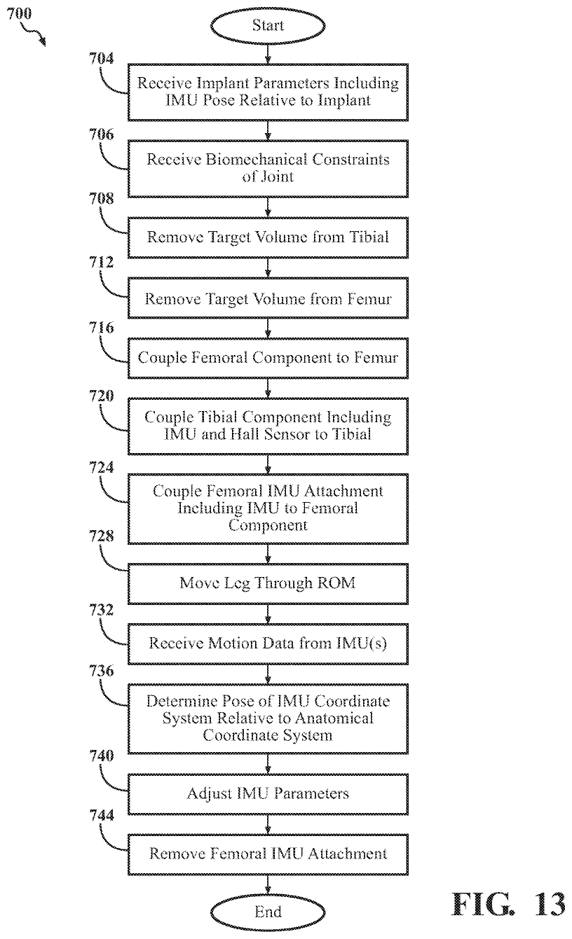

With reference to FIG. 12, a fourth exemplary method 600 according to the teachings of the present disclosure is depicted. At 604, the navigation controller 26 may receive implant parameters including the physical dimensions and geometrical profile of the implant 35 including the tibial component 106 and the femoral component 104 and pose of the IMU 64 relative to the tibial component 106. At 608, the navigation controller 26 may receive at least one medical image of the femur F and the tibia T. At 612, the navigation controller 26 may receive surgical plan data including a planned pose of the femoral component 104 in the anatomical coordinate system and a planned pose of the tibial component 106 in the anatomical coordinate system. At 616, the surgeon may couple the tracker 44, 46 to the femur F and the tibia T, respectively. At 620, the navigation controller 26 may register the global coordinate system to the anatomical coordinate system. During 612, the planned poses may instead be in the global or image coordinate system, and the navigation controller 26 may determine the planned poses in the anatomical coordinate system after registering the global/image coordinate system to the anatomical coordinate system as described herein.

At 624, the navigation controller 26 may receive femur tracking data which may be used to determine a pose of the femur F. At 628, the tibia T is resected according to the surgical plan. At 632, the femur F is resected according to the surgical plan. During both 628 and 632, the navigation controller 26 may direct the manipulator 12 and/or surgeon to cut the femur F and tibia T according to the surgical plan and track the manipulator and/or surgical instrument while the femur F and tibia T are being cut. If a deviation from the surgical plan is detected by the navigation controller 26, the surgical plan data can be updated based on the deviation. At 636, the manipulator 12 and/or surgeon may couple the femoral component 104 to the femur. At 640, the manipulator 12 and/or surgeon may couple the tibial component to the tibia T.

At 644, the navigation controller 26 may receive data from the IMU 64 corresponding to the acceleration and rotation of the IMU 64 in the sensor coordinate system. At 648, the navigation controller 26 may determine the pose of the sensor coordinate system relative to the anatomical coordinate system based on the planned pose of the tibial component 106. More specifically, since the pose of the sensor coordinate system is known relative to the physical parameters and geometrical profile of the tibial component 106, and the pose of the femoral component 106 relative to the anatomical coordinate system is also known from the surgical plan data, the pose of the sensor coordinate system can be registered to the anatomical coordinate system. At 652, the navigation controller 26 may adjust the parameters of the IMU 64 such that the sensor readings from the IMU 64 are output relative to the anatomical coordinate system and the method 500 may end.

With reference to FIG. 13, a fifth exemplary method 700 according to the teachings of the present disclosure is depicted. At 704, the navigation computer 26 may receive implant parameters including femoral component 104 and tibial component 106 parameters. For example the navigation computer 26 may receive a pose of an IMU and/or a pose of a hall-effect sensor relative to the tibial component 106. At 706, the navigation computer 26 may receive biomechanical constraints of the joint. At 708, the manipulator 12 and/or surgeon removes the target volume from the tibial T. At 712, the manipulator 12 and/or surgeon removes the target volume from the femur F. At 716, manipulator 12 and/or surgeon couples the femoral component 104 to the femur F. At 720, the manipulator 12 and/or surgeon couples the tibial component 106 including the IMU 64 and the hall effect sensor 62 to the tibia T. At 724, the manipulator 12 and/or surgeon couples the femoral IMU attachment 128 including the auxiliary IMU 134 to the femoral component 104.

At 728, the surgeon guides the leg through a predefined range of movement (ROM). At 732, the navigation computer 26, captures motion data from the IMU 64 coupled to the tibial component 16 and motion data from the auxiliary IMU 134 included in the femoral IMU attachment 128. At 736, the navigation computer 26 may determine the pose of the sensor coordinate system relative to the anatomical coordinate system based on the sensor readings from the IMU 64, the auxiliary IMU 134, and the hall effect sensor 62 as the leg is guided through the predefined ROM.

In some embodiments, the femoral IMU attachment 128 houses a magnet arranged to provide information to the hall effect sensor 62 of the tibial component 106. The magnet may be at least one of the plurality of magnets 132. In such an embodiment, at 724, the femoral IMU attachment 128 including the magnet is coupled to the femoral component 104. Alternatively, the tibial component 106 may house the magnet and the femoral IMU attachment 128 can be ignored. In either case, at 732, the navigation computer 26 captures motion data from the IMU 64 coupled to the tibial component 16 and magnetic data from the hall effect sensor 62 corresponding to a magnitude of a magnetic field associated with the magnet. At 736, the navigation computer 26 may determine the pose of the sensor coordinate system relative to the anatomical coordinate system based on the sensor readings from the IMU 64 and the magnetic data from the hall effect sensor 62 as the leg is guided through the predefined ROM.

An exemplary system and method for determining joint angle(s) based on IMU sensor readings is provided the article entitled “IMU-Based Joint Angle Measurement for Gait Analysis” by Seel et al, published in Sensors (2014), which is hereby incorporated by reference. Seel utilizes IMU sensor readings and biomechanical constraints of the joint to determine joint angle(s). Because the surgeon guides the leg through the predefined ROM (i.e. a known movement), the difference between the sensor readings from the IMU 64 and the auxiliary IMU 134, and the sensor readings expected when a leg is guided through the same predefined ROM as discussed by Seel et al, can be determined by the navigation controller 26. This difference can be used to determine the pose of the sensor coordinate system relative to the anatomical coordinate system.

At 740, the navigation controller 26 may adjust the parameters of the IMU 64 such that the sensor readings from the IMU 64 are output relative to the anatomical coordinate system. At 744, the surgeon removes the femoral IMU attachment 128 and the method 700 may end.

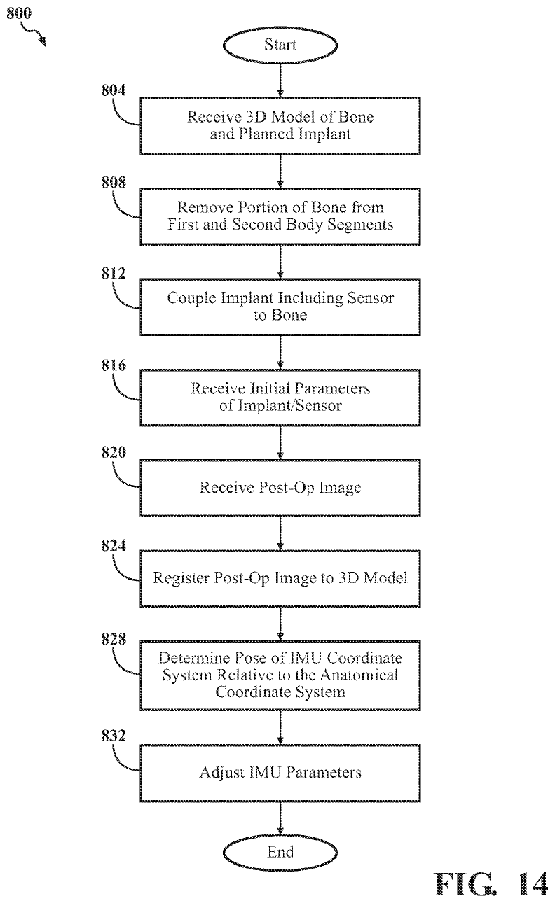

With reference to FIG. 14, a sixth exemplary method 800 according to the teachings of the present disclosure is depicted. At 804, the navigation controller 26 may receive 3D image data (e.g. a 3D model of at least a portion of the anatomy of the patient) from a CT or MRI machine. For example, the navigation controller 26 may receive a 3D model of the femur F and the tibia T of the patient, with the 3D model including a representation of the patient's femur F and tibia T in an image coordinate system associated with the CT or MRI machine used to capture the 3D image data. The navigation controller 26 may then determine the anatomical coordinate system based on the 3D image data (e.g. 3D model) via any suitable method. As such, the navigation controller 26 will know the pose of the anatomical coordinate system relative to the image coordinate system.

At 808, the manipulator 12 and/or surgeon may remove a target volume of the tibia T and a target volume of the femur F according to the surgical plan. At 812, the manipulator 12 and/or surgeon may couple the implant 35 including the femoral component 106 and the tibial component 104 to the femur F and the tibia T, respectively. At 816, the navigation controller 26 may receive implant parameters including the physical dimensions and geometrical profile of the implant 35 including the tibial component 106 and the femoral component 104 and pose of the IMU 64 relative to the tibial component 106 and/or the femoral component 104. For example, the navigation controller 26 may receive the physical dimensions and geometrical profile of the tibial component 106, as well as the pose of the sensor coordinate system relative to said physical dimensions and geometrical profile of the tibial component 106.

At 820, the navigation controller 26 may receive at least one medical image of the patient (e.g. an x-ray image), the at least one medical image generally including the femur F and the tibia T of the patient, as well as the implant 35. The at least one medical image is generally a 2D image. At 824, the navigation controller 26 may register the at least one medical image to the 3D image data such that the femur F and the tibia T depicted in the at least one medical image is aligned with the femur F and the tibia T represented by the 3D image data. For example, the navigation controller 26 may utilize shape-matching and/or contour-matching algorithm(s) in which the shapes/contours of the femur F and tibia T in the at least one medical image are overlaid to match the shapes/contours of the respective femur F and tibia T in the 3D image data.

At 828, the navigation controller 26 may determine the pose of the sensor coordinate system relative to the anatomical coordinate system based on at least one of the pose of the implant 35 and the pose of the identification feature in the at least one image. More specifically, since the pose of the sensor coordinate system is known relative to the physical parameters and geometrical profile of the implant 35, and the pose of the implant 35 relative to the anatomical coordinate system is also known from the at least one image, the pose of the sensor coordinate system can be registered to the anatomical coordinate system. The pose of the implant 35 relative to the anatomical coordinate system is known from the at least one image because (1) the pose of the anatomical coordinate system is known relative to the image coordinate system of the 3D model and (2) the pose of the implant 35 relative to the image coordinate system is known based on the registration performed at 824. At 832, the navigation controller 26 may adjust the parameters of the IMU 64 such that the sensor readings from the IMU 64 are output relative to the anatomical coordinate system and the method 500 may end.

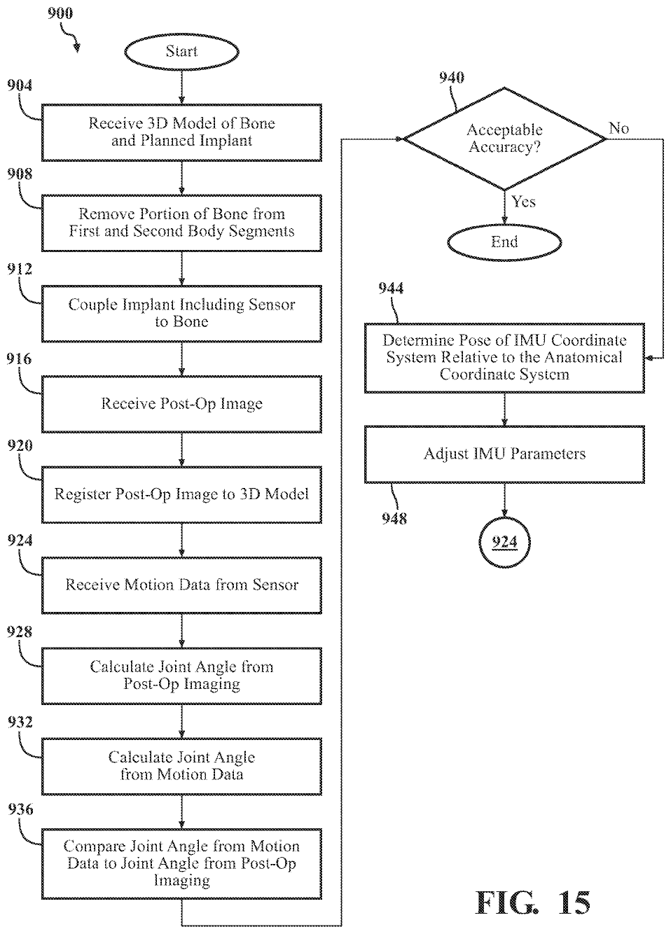

With reference to FIG. 15, a seventh exemplary method 900 according to the teachings of the present disclosure is depicted. At 904, the navigation controller 26 may receive 3D image data (e.g. a 3D model of at least a portion of the anatomy of the patient) from a CT or MRI machine. For example, the navigation controller 26 may receive a 3D model of the femur F and the tibia T of the patient, with the 3D model including a representation of the patient's femur F and tibia T in an image coordinate system associated with the CT or MRI machine used to capture the 3D image data. The navigation controller 26 may then determine the anatomical coordinate system based on the 3D image data (e.g. 3D model) via any suitable method. As such, the navigation controller 26 will know the pose of the anatomical coordinate system relative to the image coordinate system.

At 908, the manipulator 12 and/or surgeon may remove a target volume of the tibia T and a target volume of the femur F according to the surgical plan. At 912, the manipulator 12 and/or surgeon may couple the implant 35 including the femoral component 106 and the tibial component 104 to the femur F and the tibia T, respectively. At 916, the navigation controller 26 may receive at least one medical image of the patient (e.g. an x-ray image), the at least one medical image generally including the femur F and the tibia T of the patient, as well as the implant 35. The at least one medical image is generally a 2D image. At 920, the navigation controller 26 may register the at least one medical image to the 3D image data such that the femur F and the tibia T depicted in the at least one medical image is aligned with the femur F and the tibia T represented by the 3D image data. For example, the navigation controller 26 may utilize shape-matching and/or contour-matching algorithm(s) in which the shapes/contours of the femur F and tibia T in the at least one medical image are overlaid to match the shapes/contours of the respective femur F and tibia T in the 3D image data.

At 924, the navigation controller 26 may receive motion data from the IMU 64 corresponding to the acceleration and rotation of the IMU 64 as detected by the sensors included in the IMU 64. At 928, the navigation controller 26 may determine at least one joint angle based on the registration performed at 920. For example, the navigation controller 26 may use the relative pose of the femur F and the tibia T as depicted in the at least one medical image. As the pose of the femur F and the tibia T are known in the anatomical coordinate system based on the registration at 920, the joint angle is thus known by the navigation controller 26 in the anatomical coordinate system. At 932, the navigation controller 26 may determine at least one joint angle based on the motion data from the IMU 64 using an algorithm.

At 936, the navigation controller 26 may compare the joint angle based on the motion data to the joint angle based on the at least one medical image. At 940, the navigation controller 26 may determine whether the difference between the joint angle determined based on the motion data and the joint angle determine based on the at least one medical image is within a threshold range of accuracy of each other. If so, the method 900 may end; otherwise, the method 900 may continue at 944. The navigation controller 26 may utilize other data, such as a predetermined sensor output corresponding to the joint angle from the at least one medical image, to determine what data is expected from the IMU 64 when the joint is at the joint angle determined from the at least one medical image. In other words, if the IMU 64 is already outputting raw data consistent with this expected data, then the pose of the sensor coordinate system is already in alignment with the anatomical coordinate system such that the IMU 64 outputs data relative to the anatomical coordinate system.

At 944, the navigation controller 26 may determine the pose of the sensor coordinate system relative to the anatomical coordinate system based on the difference between the joint angle determined based on the motion data and the joint angle determine based on the at least one medical image. More specifically, since the joint angle is known relative to the anatomical coordinate system based on the at least one medical image and the process at 920, the navigation controller knows what joint angle should be output by the IMU 64. The navigation controller 26 may thus determine the pose of the sensor coordinate system relative to the anatomical coordinate system based on the knowledge of what joint angle should be output by the IMU 64 and what is actually being output by the IMU 64. The navigation controller 26 may utilize other data, such as a predetermined sensor output corresponding to the joint angle from the at least one medical image, to determine a transform capable of bringing the joint angle according to the motion data into alignment with the joint angle according to the at least one medical image. At 948, the navigation controller 26 may adjust the parameters of the IMU 64 such that the sensor readings from the IMU 64 are output relative to the anatomical coordinate system and the method 900 may proceed to 924.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements.

As used herein, the phrase at least one of A, B, and C should be construed to mean a logical (A OR B OR C), using a non-exclusive logical OR, and should not be construed to mean “at least one of A, at least one of B, and at least one of C.” The term subset does not necessarily require a proper subset. In other words, a first subset of a first set may be coextensive with (equal to) the first set.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from clement A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

Some or all hardware features of a module may be defined using a language for hardware description, such as IEEE Standard 1364-2005 (commonly called “Verilog”) and IEEE Standard 1076-2008 (commonly called “VHDL”). The hardware description language may be used to manufacture and/or program a hardware circuit. In some implementations, some or all features of a module may be defined by a language, such as IEEE 1666-2005 (commonly called “SystemC”), that encompasses both code, as described below, and hardware description.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

1. A method for registering a sensor coordinate system of a first sensor of an implant to an anatomical coordinate system using a surgical system, the implant including a second sensor, the surgical system including a surgical navigation system, the first sensor having a predetermined spatial relationship relative to the implant, the implant being coupled to a bone of a patient, the method comprising:
receiving at least one medical image defining the anatomical coordinate system, the at least one medical image including a first body segment and a second body segment forming a joint; and
after the implant has been coupled to the first body segment and the second body segment forming the joint, while the first body segment and the second body segment are in a first pose:
obtaining motion data from at least one of the first sensor and the second sensor, and
obtaining navigation data of the first body segment and the second body segment with the surgical navigation system;
determining a first angle of the joint based on the motion data;
determining a second angle of the joint based on the surgical navigation system;
comparing the first angle to the second angle; and
selectively adjusting one or more parameters of the first sensor so that the first angle falls within a threshold of the second angle.

2. The method of claim 1, wherein obtaining the second angle with the surgical navigation system includes determining a pose of the first body segment and a pose of the second body segment with the surgical navigation system.

3. The method of claim 2, where the first body segment is a femur and the second body segment is a tibia.

4. The method of claim 1, wherein the first angle and the second angle include at least one of a flexion/extension (F/E) angle, an internal/external (I/E) rotation angle and a varus/valgus (V/V) angle.

5. The method of claim 1, further comprising coupling a first tracking element to the first body segment and coupling a second tracking element to the body second segment, the surgical navigation system using the first tracking element and the second tracking element in determining a pose of the first body segment and a pose of the second body segment, the pose of the first body segment and the pose of the second body segment being used to calculate the first angle.

6. The method of claim 1, wherein obtaining the second angle of the joint of the patient with the surgical navigation system includes identifying a center of the joint.

7. The method of claim 1, wherein the surgical navigation system includes a camera configured to autonomously identify coordinates of the joint and at least one of a pose of the first body segment and a pose of the second body segment without a tracking element coupled to either the first body segment or the second body segment.

8. The method of claim 1, wherein the sensor coordinate system includes an x-axis, a y-axis, and a z-axis, and wherein the first sensor is configured to measure a roll about the x-axis of the sensor coordinate system, a pitch about the y-axis of the sensor coordinate system, and a yaw about the z-axis of the sensor coordinate system.

9. The method of claim 1, wherein the sensor coordinate system includes at least one axis and wherein adjusting the one or more parameters includes:
determining at least one anatomical axis defined by the anatomical coordinate system based on the motion data in the anatomical coordinate system and the at least one medical image; and
determining a relationship between the at least one anatomical axis and the at least one axis of the sensor coordinate system; and
adjusting the one or more parameters based on the relationship.

10. The method of claim 1, further comprising coupling the implant to at least one of the first body segment and the second body segment.

11. The method of claim 1, wherein the first body segment is a femur and the second body segment is a tibia, the implant is a knee implant including a femoral component and a tibial component, the femoral component includes a plurality of magnets, and the tibial component includes the first sensor and the second sensor, the first sensor configured to generate inertial data representative of a pose of the tibia, the second sensor configured to generate a magnetic data representative of a pose of the plurality of magnets relative to the second sensor, the motion data including the inertial data and the magnetic data, the method further comprising:
determining a pose of the tibia based on the inertial data;
determining a pose of the femur based on the magnetic data and the inertial data; and
determining the first angle based on a pose of the femur and the pose of the tibia.

12. The method of claim 11, wherein the second sensor is a hall effect sensor.

13. The method of claim 1, wherein the first sensor and the second sensor are inertial measurement units, the implant is a knee implant including a femoral component and a tibial component, the first sensor being disposed on the tibial component, the second sensor being disposed on the femoral component.

14. The method of claim 1, further comprising correlating motion data obtained in the first pose to navigation data obtained in the first pose.

15. The method of claim 1, further comprising setting current implant parameters as final implant parameters in response to the first sensor being within the threshold of the second angle.

16. A system comprising:
an implant including a sensor defining a sensor coordinate system including at least one sensor axis, the implant configured to be fixed to a first bone of a patient and the sensor having a first fixed spatial relationship relative to the implant, wherein the sensor is configured to output inertial data of the implant;
a patient tracking element configured to couple to the patient;
a surgical instrument configured to couple to the implant, the surgical instrument including an instrument tracking element; and
a navigation system configured to:
receive an image of the patient defining an anatomical coordinate system;
register the patient in a global coordinate system to the anatomical coordinate system;
track a pose of the surgical instrument in the global coordinate system,
determine a pose of the implant based on the pose of the surgical instrument in the global coordinate system,
determine the pose of the implant in the anatomical coordinate system; and
selectively adjust one or more parameters of the sensor to register the sensor coordinate system to the anatomical coordinate system.

17. The system of claim 16, wherein the navigation system determines the pose of the implant in the global coordinate system based on the pose of the surgical instrument in the global coordinate system and a spatial relationship between the surgical instrument and the implant.

18. The system of claim 16, wherein the surgical instrument includes a robotic manipulator configured to guide the implant relative to the patient.

19. The system of claim 16, wherein the sensor includes an inertial measurement unit configured to output the inertial data which corresponds to the pose of the implant.

20. The system of claim 19, wherein the sensor coordinate system includes an x-axis, a y-axis, and a z-axis, and wherein the inertial measurement unit is configured to measure a roll about the x-axis of the sensor coordinate system, a pitch about the y-axis of the sensor coordinate system, and a yaw about the z-axis of the sensor coordinate system.

21-55. (canceled)
